(19) United States
(12) Patent Application Publication
(10) Pub. No.: US 2007/0053860 A1
(43) Pub. Date: Mar. 8, 2007

(54) Short-chain enol esters as odiferous substance precursors

(76) Inventors: Marcus Eh, Holzminden (DE); Johannes Panten, Hoxter (DE); Heinz-Jurgen Bertram, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/575,781

(22) PCT Filed: Oct. 13, 2004
(86) PCT No.: PCT/EP04/52520

§ 371 Date: Apr. 14, 2006

(30) Foreign Application Priority Data
Oct. 16, 2003 (DE) 103 48 062.5

(51) Int. Cl. A61K 8/37

(52) U.S. Cl. 424/70.2; 424/401

(57) ABSTRACT

The use of a compound of the formula I 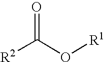 in which
R¹ is the radical (a) of the enol form of an aldehyde having 6 or more C atoms or (b) of a ketone having 10 or more C atoms and
R² is an (a) branched or unbranched C₁ to C₄ alkyl group or (b) branched or unbranched C₂ to C₄ alkylene group, as a fragrance precursor.

The present invention relates to the use of certain short-chain enol esters as precursors for the controlled release of organoleptically active aldehydes or ketones. Specifically, the aldehydes or ketones released are fragrances. The fragrance precursors are obtainable by reaction of aldehydes or ketones with carboxylic acid anhydrides.

The invention moreover relates to methods for the release of a fragrance substance as well as (a) cosmetic, washing and/or cleaning formulations and (b) perfume oils which comprise the fragrance precursor according to the invention.

The main procedure for perfuming consumer articles is that of mixing the perfume oil comprising fragrances directly with the product. A problem which occurs here is that numerous substances, and in this context in particular also aldehydes and in some cases also ketones, are unstable under the given conditions, which leads to partial or complete decomposition of these molecules in the course of storage. The consequence of this is that all substances which suffer from the problem described above can be perceived sensorially only weakly or not at all in the end product. In individual cases, this can lead to an unacceptable change in the overall smell impression of the composition.

U.S. Pat. No. 5,649,979 discloses enol esters of type (II) 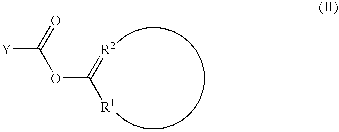
wherein Y represents an unbranched or branched, saturated or unsaturated C₇ to C₂₄ radical, and on the one hand R¹═H and R² represents the radical of a fragrance aldehyde R²CHO, or on the other hand R¹ and R² represent radicals of a fragrance ketone:

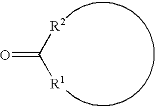

The enol esters described release the aldehyde or the ketone slowly and are claimed for use in washing powders and fabric softeners.

U.S. Pat. No. 6,207,857 discloses enol esters of the formula (III) 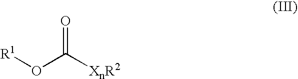
wherein R¹ represents the enol form of an aldehyde defined in more detail or of a ketone, X e.g. represents a hydrocarbon radical defined in more detail, R² e.g. represents a carbocyclic or heterocyclic radical defined in more detail or COOY, wherein Y is an H atom, a metal or R³, wherein R³ is the radical of an alcohol or phenol or has the same definition as R¹, and wherein n is 0 or 1. The compounds of the formula (III) are said to be virtually odourless and, under activation conditions, to release one or more compounds which have organoleptic and/or antimicrobial properties. The fragrance precursors of the formula (III) furthermore release the active molecules relatively slowly.

U.S. Pat. No. 6,479,682 moreover discloses protected hydroxy esters of the formula (IV) 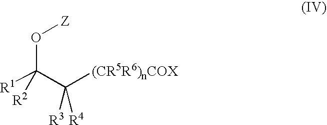
wherein the substituents have meanings defined in more detail. The compounds of the formula (IV) dissociate in 2 stages; firstly, the “protective group” Z is split off and a hydroxy ester is formed, and in a second step the hydroxy ester cyclizes to form the corresponding lactone and during this procedure splits off an alcohol, aldehyde or ketone. The compounds of the formula (IV) are said to be virtually odourless and here also the release of the active molecules takes place slowly.

U.S. Pat. No. 6,262,287 discloses siloxanes of the formula (Va) and (Vb) 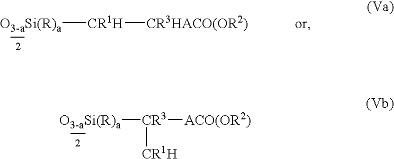
wherein the radicals have a meaning defined in more detail. The compounds of the formulae (Va) and (Vb) disclosed are virtually odourless and are cleaved by contact with skin or by lipases such that fragrance alcohols, or aldehydes or ketones are released.

The prior art acknowledged above shows that a number of fragrance precursors which bond the enol form of an aldehyde or ketone by means of an ester functionality and, after activation, release the aldehyde or the ketone slowly, i.e. over a period of several hours or days, are known. It is a disadvantage that the fragrance precursors such as are disclosed in U.S. Pat. No. 5,649,979, U.S. Pat. No. 6,207,857, U.S. Pat. No. 6,479,682 and U.S. Pat. No. 6,262,287 are not suitable for virtually spontaneous release of an aldehyde or ketone after activation.

It was therefore the primary object of the present invention to provide the use of compounds as fragrance precursors which have a very much higher storage stability than the corresponding aldehydes or ketones (bonded via their enol form) after incorporation into a product and which, after activation (cleavage), release the aldehydes or ketones virtually spontaneously.

According to the invention, this object is achieved by the use of a compound of the formula I 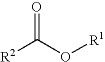
in which
R¹ is the radical (a) of the enol form of an aldehyde having 6 or more C atoms or (b) of a ketone having 10 or more C atoms
and
R² is an (a) branched or unbranched C₁ to C₄ alkyl group or (b) branched or unbranched C₂ to C₄ alkylene group,
as a fragrance precursor.

In this context, R² can be, in particular:
(a) methyl, ethyl, n-propyl, iso-propyl, n-butyl, sec-butyl, iso-butyl or tert-butyl. To this extent, however, the alkyl radicals methyl, ethyl, n-propyl and iso-butyl are preferred, and the alkyl radicals methyl, ethyl and iso-butyl are particularly preferred.
(b) ethenyl, methylethenyl, 1-propenyl, 2-propenyl, 2-methyl-1-propenyl, 1-methyl-1-propenyl, 1-butenyl, 3-butenyl. To this extent, however, the alkylene radicals ethenyl, methylethenyl, 1-propenyl, 2-methyl-1-propenyl and 1-methyl-1-propenyl are preferred, and the alkylene radicals ethenyl, methylethenyl and 1-propenyl are particularly preferred.

Surprisingly, in contrast to the corresponding aldehydes or ketones, the compounds of the formula (I) to be used according to the invention as fragrance precursors have a good storage stability in (a) acidic, oxidative media and (b) in alkaline media having a water content of ≦10%. The storage stability in acidic, oxidative media is surprising since in U.S. Pat. No. 3,923,247 and Gerasimovich, T. B. et al. Natural'nykh Dushistykh Veshchestv, 1965, 38-42 it is reported that enol acetates are hydrolysed in the presence of 6N H₂SO₄.

The invention also relates to a method for the release of a fragrance, having the following steps:
provision of a compound of the formula I 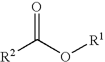
   in which
R¹ is the radical (a) of the enol form of an aldehyde having 6 or more C atoms or (b) of a ketone having 10 or more C atoms
and
R² is an (a) branched or unbranched C₁ to C₄ alkyl group or (b) branched or unbranched C₂ to C₄ alkylene group,
preparation of a formulation which comprises the compound of the formula I and a medium such that the compound of the formula I is stable in the formulation,
treatment of the formulation such that the compound of the formula I dissociates and releases the fragrance.

In accordance with the advantageous storage stabilities, the medium is advantageously (a) acidic and oxidative, or it is (b) alkaline and has a water content of ≦10 wt. %, based on the total weight of the medium.

The treatment of the formulation then preferably comprises a step in which
in case (a) the pH of the formulation is raised to a value of ≧8.5
or
in case (b) the water content of the formulation is raised to >10 wt. %.

It is ensured in this manner that the compound of the formula I dissociates spontaneously and releases the fragrance.

The formulation itself is preferably

in case (a) chosen from the group which consists of: developer composition for a permanent hair-colouring composition, permanent wave fixing composition, bleaching cream, acne cream, sanitary cleaner and surface cleaner

or

in case (b) chosen from the group which consists of: liquid detergents for packages in water-soluble film, deodorant or antiperspirant sticks and soaps.

This aspect is explained in detail below.

For case (a), the activation and thus virtually spontaneous dissociation of the compounds of the formula (I) to be used as fragrance precursors and the associated release of an aldehyde or ketone thus preferably take place by the direct raising of the pH into the alkaline range with a resulting pH of ≧8.5. For case (b), the activation preferably takes place by addition of water, and as a consequence thereof a virtually spontaneous dissociation of the compounds of the formula (I) to be used as fragrance precursors takes place.

The present invention also relates to cosmetic, washing and/or cleaning formulations comprising or consisting of:
a compound of the formula I 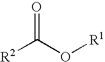
   in which
R¹ is the radical (a) of the enol form of an aldehyde having 6 or more C atoms or (b) of a ketone having 10 or more C atoms
and
R² is an (a) branched or unbranched C₁ to C₄ alkyl group or (b) branched or unbranched C₂ to C₄ alkylene group
and
a medium comprising further or the further formulation constituents,
   wherein the content of the compound of the formula I in the formulation is less than 1 wt. %, based on the total weight of the formulation, and wherein the medium is chosen such that the compound of the formula I is stable in the formulation.

In respect of the compound of the formula I and its preferred embodiment, the statements made above again apply.

Advantageously—and for the abovementioned reasons—in a formulation according to the invention the medium is either (a) acidic and oxidative, or it is (b) alkaline and has a water content of ≦10 wt. %, based on the total weight of the medium.

The formulation is then preferably in case (a) chosen from the group which consists of: developer composition for a permanent hair-colouring composition, permanent wave fixing composition, bleaching cream, acne cream, sanitary cleaner and surface cleaner

or

in case (b) chosen from the groups which consists of liquid detergents for packages in water-soluble film, deodorant or antiperspirant sticks and soaps.

Advantageously, in a formulation according to the invention,

(a) the compound of the formula I is dispersed or dissolved in the medium

and/or

(b) the compound of the formula I is employed as a constituent of a perfume oil (in this context, see below) which is dispersed or dissolved in the medium.

In this context, in case (b) the perfume oil optionally is (i) adsorbed on a carrier substance, (ii) microencapsulated or (iii) spray-dried, or it is employed (iv) as an inclusion complex or (v) extrusion product, or (vi) coated.

The invention also relates to a perfume oil itself, where this comprises:
a compound of the formula I

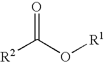

in which
R¹ is the radical (a) of the enol form of an aldehyde having 6 or more C atoms or (b) of a ketone having 10 or more C atoms
and
R² is an (a) branched or unbranched C₁ to C₄ alkyl group or (b) branched or unbranched C₂ to C₄ alkylene group

1and
one or more fragrances,
   wherein the content of the compounds of the formula I in the perfume oil is at least 0.1 wt. %, based on the total weight of the perfume oil.

In this context, perfume oil according to the invention optionally is (i) adsorbed on a carrier substance, (ii) microencapsulated or (iii) spray-dried, or it is employed (iv) as an inclusion complex or (v) extrusion product, or (vi) coated.

Further detailed statements regarding the perfume oils according to the invention which can be employed in particular for the preparation of formulations according to the invention, for carrying out methods according to the invention and in the use according to the invention are found below. It goes without saying that all the explanations given in the context of the present text regarding the compounds of the formula (I) (preferably) to be used relate to all the aspect of the invention (use, methods, formulation, perfume oil etc.).

Non-limiting examples of aldehydes which are preferably released after cleavage of a compound of the formula (I) to be used according to the invention as a fragrance precursor may be mentioned in the following:

phenylacetaldehyde, p-methylphenylacetaldehyde, p-isopropylphenylacetaldehyde, methylnonylacetaldehyde, phenylpropanal, 3-(4-t-butylphenyl)-2-methylpropanal (Lilial), 3-(4-t-butylphenyl)-propanal (bourgeonal), 3-(4-methoxyphenyl)-2-methylpropanal (canthoxal), 3-(4-isopropylphenyl)-2-methylpropanal (cymal), 3-(3,4-methylenedioxyphenyl)-2-methylpropanal (Helional), 3-(4-ethylphenyl)-2,2-dimethylpropanal (floralozone), phenylbutanal, 3-methyl-5-phenylpentanal, hexanal, trans-2-hexenal, cis-hex-3-enal, heptanal, cis-4-heptenal, 2-ethyl-2-heptenal, 2,6-dimethyl-5-heptenal (melonal), 2,4-heptadienal, octanal, 2-octenal, cis-5-octenal, 3,7-dimethyloctanal, 3,7-dimethyl-2,6-octadien-1-al, 3,7-dimethyl-2,6-octadien-3-al, 3,7-dimethyl-6-octenal (citronellal), 3,7-dimethyl-7-hydroxyoctan-1-al (hydroxycitronellal), nonanal, cis-6-nonenal, 2,4-nonadienal, 2,6-nonadienal, decanal, 2-methyidecanal, 4-decenal, 9-decenal, 2,4-decadienal, undecanal, 2-methyldecanal, 2-methylundecanal, 2,6,10-trimethyl-9-undecenal (adoxal), undec-10-enylaldehyde, undec-8-enanal, dodecanal, tridecanal, tetradecanal, anisaldehyde, cinnamaldehyde, α-amylcinnamaldehyde, α-hexylcinnamaldehyde, methoxycinnamaldehyde, isocyclocitral, citronellyloxyacetaldehyde, cortexaldehyde, cuminaldehyde, cyclamenaldehyde, florhydral, heliotropin, hydratropaaldehyde, vanillin, ethylvanillin, benzaldehyde, p-methylbenzaldehyde, 3,4-dimethoxybenzaldehyde, 3- and 4-(4-hydroxy-4-methylpentyl)-3-cyclohexene-1-carboxaldehyde (Lyral), 2,4-dimethyl-3-cyclohexene-1-carboxaldehyde (Triplal), I-methyl-3-(4-methylpentyl)-3-cyclohexenecarboxaldehyde (vernaldehyde) or p-methylphenoxyacetaldehyde (Xi aldehyde).

Non-limiting examples of ketones which are released after cleavage of a compound of the formula (I) to be used according to the invention as a fragrance precursor may be mentioned in the following:

α-damascone, β-damascone, δ-damascone, β-damascenone, muscone, 6,7-dihydro-1,1,2,3,3-pentamethyl-4(5h)-indanone (Cashmeran), cis-jasmone, dihydrojasmone, α-ionone, β-ionone, dihydro-β-ionone, γ-methylionone, α-iso-methylionone, 4-(3,4-methylenedioxyphenyl)butan-2-one, 4-(4-hydroxyphenyl)butan-2-one, methyl β-naphthyl ketone, methyl cedryl ketone, 6-acetyl-1,1,2,4,4,7-hexamethyltetralin (Tonalid), l-carvone, 5-cyclohexadecen-1-one, acetophenone, decatone, p-hydroxyphenylbutan-2-one, 2-[2-(4-methyl-3-cyclohexenyl-1-yl)propyl]cyclopentan-2-one, 2-sec-butyl-cyclohexanone, β-dihydroionone, allylionone, α-irone, α-cetone, α-irisone, acetanisole, geranylacetone, 1-(2-methyl-5-isopropyl-2-cyclohexenyl)-1-propanone, acetyldiisoamylene, methylcyclocitrone, 4-t-pentylcyclohexanone, p-t-butylcyclohexanone, o-t-butylcyclohexanone, ethyl amyl ketone, ethyl pentyl ketone, menthone, methyl-7,3-dihydro-2h-1,5-benzodioxepin-3-one or fenchone.

The radicals R¹ in formula I result of course from the aldehydes and ketones mentioned—after conversion into their particular enol form.

The virtually spontaneous release of an aldehyde or ketone after cleavage of the compounds of the formula (I) according to the invention to be used as fragrance precursors can be used for treatment (e.g. fragrancing) of an abundance of substrates, such as e.g. hair, human skin, laundry and hard surfaces.

Examples of fragrances with which the compounds of the formula (I) to be used according to the invention as fragrance precursors can advantageously be combined are to be found e.g. in S. Arctander, Perfume and Flavor materials, vol. I and II, Montclair, N. J., 1969, author and publisher, or K. Bauer, D. Garbe and H. Surburg, Common Fragrance and Flavor Materials, 3rd ed., Wiley-VCH, Weinheim 1997.

There may be mentioned in detail:

extracts from natural raw materials, such as essential oils, concretes, absolutes, resins, resinoids, balsams, tinctures, such as e.g. amber tincture; amyris oil; angelica seed oil; angelica root oil; aniseed oil; valerian oil; basil oil; tree moss absolute; bay oil; artemisia oil; benzoin resin; bergamot oil; beeswax absolute; birch tar oil; bitter almond oil; bean leaf oil; buchu leaf oil; cabreuva oil; cade oil; calamus oil; camphor oil; cananga oil; cardamom oil; cascarilla oil; cassia oil; cassia absolute; castoreum absolute; cedar leaf oil. cedar wood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; Eucalyptus citriodora oil; eucalyptus oil; fennel oil; spruce needle oil; galbanum oil; galbanum resin, geranium oil; grapefruit oil; guaiac wood oil; gurjunene balsam; gurjunene balsam oil; helichrysum absolute; helichrysum oil; ginger oil; iris root absolute; iris root oil; jasmine absolute; calamus oil; camomile oil blue; camomile oil Roman; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon grass oil; lovage oil; lime oil distilled; lime oil pressed; linaloa oil; Litsea cubeba oil; bay leaf oil; mace oil; marjoram oil; mandarin oil; massoi bark oil; mimosa absolute; musk seed oil; musk tincture; muscatel sage oil; nutmeg oil; myrrh absolute; myrrh oil; myrtle oil; clove leaf oil; clove blossom oil; neroli oil; olibanum absolute; olibanum oil; opopanax oil; orange blossom absolute; orange oil; oregano oil; palmarosa oil; patchouli oil; perilla oil; Peru balsam oil; parsley leaf oil; parsley seed oil; petitgrain oil; peppermint oil; pepper oil; pimento oil; pine oil; poley oil; rose absolute; rose wood oil; rose oil; rosemary oil; sage oil Dalmatian; sage oil Spanish; sandalwood oil; celery seed oil; spike lavender oil; star aniseed oil; styrax oil; tagetes oil; fir needle oil; tea tree oil; terpentine oil; thyme oil; tolu balsam oil; tonka absolute; tuberose absolute; vanilla extract; violet leaf absolute; verbena oil; vetiver oil; juniper berry oil; wine yeast oil; wormwood oil; wintergreen oil; ylang oil; hyssop oil; civet absolute; cinnamon leaf oil; cinnamon bark oil; and fractions thereof or constituents isolated therefrom.

Individual fragrances from the group of the hydrocarbons, such as e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene and (E,Z)-1,3,5-undecatriene;

the aliphatic alcohols, such as e.g. hexanol; octanol; 3-octanol; 2,6-dimethylheptanol; 2-methylheptanol, 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-di-methyl-7-methoxyoctan-2-ol; 9-decenol and 10-undecenol; 4-methyl-3-decen-5-ol; the aliphatic aldehydes and 1,4-dioxacycloalken-2-ones thereof, such as e.g. hexanal; heptanal; octanal; nonanal; decanal; undecanal; dodecanal; tridecanal; 2-methyloctanal; 2-methyinonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene and citronellyloxyacetaldehyde;

the aliphatic ketones and oximes thereof, such as e.g. 2-heptanone; 2-octanone; 3-octanone; 2-nonanone; 5-methyl-3-heptanone; 5-methyl-3-heptanone oxime and 2,4,4,7-tetramethyl-6-octen-3-one; the aliphatic sulfur-containing compounds, such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate and 1-menthene-8-thiol;

the aliphatic nitriles, such as e.g. 2-nonenoic acid nitrile; 2-tridecenoic acid nitrile; 2,12-tridecenoic acid nitrile; 3,7-dimethyl-2,6-octadienoic acid nitrile and 3,7-dimethyl-6-octenoic acid nitrile;

the aliphatic carboxylic acids and esters thereof, such as e.g. (E)- and (Z)-3-hexyenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynate; methyl 2-nonynate; allyl 2-isoamyloxyacetate and methyl 3,7-dimethyl-2,6-octadienoate;

the acyclic terpene alcohols, such as e.g. citronellol; geraniol; nerol; linalool; lavadulol; nerolidol; farnesol; tetrahydrolinalool; tetrahydrogeraniol; 2,6-di-methyl-7-octen-2-ol; 2,6-dimethyloctan-2-ol; 2-methyl-6-methylen-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol and 2,6-dimethyl-2,5,7-octatrien-1-ol; and formates, acetates, propionates, isobutyrates, butyrates, isovalerates, pentanoates, hexanoates, crotonates, tiglinates and 3-methyl-2-butenoates thereof;

the acyclic terpene aldehydes and ketones, such as e.g. geranial; neral; citronellal; 7-hydroxy-3,7-dimethyloctanal; 7-methoxy-3,7-dimethyloctanal; 2,6,10-trimethyl-9-undecenal and geranylacetone; and the dimethyl and diethyl acetals of geranial, neral and 7-hydroxy-3.7-dimethyloctanal

the cyclic terpene alcohols, such as e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol and guaiol; and formates, acetates, propionates, isobutyrates, butyrates, isovalerates, pentanoates, hexanoates, crotonates, tiglinates and 3-methyl-2-butenoates thereof;

the cyclic terpene aldehydes and ketones, such as e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal and acetylated cedar wood oil (methyl cedryl ketone);

the cyclic alcohols, such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol and 2-isobutyl-4-methyltetrahydro-2H-pyran-4-ol;

the cycloaliphatic alcohols, such as e.g. alpha,3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol and 1-(2,2,6-trimethylcyclohexyl)hexan-3-ol;

the cyclic and cycloaliphatic ethers, such as e.g. cineol; cedryl methyl ether; cyclododecyl methyl ether; (ethoxymethoxy)cyclododecane; alpha-cedrene epoxid; 3a,6,6,9a-tetramethyldodecahydronaphtho[2,1-b]furan; 3a-ethyl-6,6,9a-trimethyldodecahydronaphtho[2, 1-b]furan; 1,5,9-trimethyl-13-oxabicyclo[10.1.0]trideca-4,8-diene; rose oxide and 2-(2,4-dimethyl-3-cyclohexen-1-yl)-5-methyl-5-(1-methyl propyl)-1,3-dioxane;

the cyclic ketones, such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentyl-cyclohexanone; 5-cyclohexadecen-1-one; 6,7-dihydro-1,1,2,3,3-pentamethyl-4(5H)-indanone; 5-cyclohexadecen-1-one; 8-cyclohexadecen-1-one; 9-cycloheptadecen-1-one; cyclopentadecanone;

the cycloaliphatic aldehydes, such as e.g. 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde and 4-(4-methyl-3-penten-1-yl)-3-cyclohexenecarbaldehyde;

the cycloaliphatic ketones, such as e.g. 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone and tert-butyl (2,4-dimethyl-3-cyclohexen-1-yl) ketone;

the esters of cyclic alcohols, such as e.g. 2-tert-butylcyclohexyl acetate; 4-tert butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- and 6-indenyl acetate; 4,7-methano-3a,4,5,6,7,7a-hexahydro-5- and 6-indenyl propionate; 4,7-methano-3a,4,5,6,7,7a-hexahydro-5- and 6-indenyl isobutyrate and 4,7-methanooctahydro-5- and 6-indenyl acetate;

the esters of cycloaliphatic carboxylic acids, such as e.g. allyl 3-cyclohexylpropionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate and ethyl 2-methyl-1,3-dioxolane-2-acetate;

the aromatic hydrocarbons, such as e.g. styrene and diphenylmethane;

the araliphatic alcohols, such as e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol and 1-(4-isopropylphenyl)ethanol;

the esters of araliphatic alcohols with aliphatic carboxylic acids, such as e.g.: benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate and 4-methoxybenzyl acetate; the araliphatic ethers, such as e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl4-phenyl-1,3-dioxane; 4,4a,5,9b-tetrahydroindeno[1,2-d]-m-dioxin and 4,4a,5,9b-tetrahydro-2,4-dimethylindeno[1,2-d]-m-dioxin;

the aromatic or araliphatic aldehydes, such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal and 2-methyl-3-(4-methylenedioxyphenyl)propanal;

the aromatic and araliphatic ketones, such as e.g. acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methylethyl)-1H-5-indenyl]ethanone and 5′,6′,7′,8′-tetrahydro-3′,5′,5′,6′,8′,8′-hexamethyl-2-acetonaphthone;

the aromatic and araliphatic carboxylic acids and esters thereof, such as e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethyl-phenyl acetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate and ethyl 3-methyl-3-phenylglycidate;

the nitrogen-containing aromatic compounds, such as e.g. 2,4,6-trinitro-1,3-dimethyl-5-tert-butylbenzene; 3,5-dinitro-2,6-dimethyl-4-tert-butylacetophenone; cinnamic acid nitrile; 5-phenyl-3-methyl-2-pentenoic acid nitrile; 5-phenyl-3-methylpentanoic acid nitrile; methyl anthranilate; methyl N-methyl-anthranilate; Schiff's bases of methyl anthranilate with 7-hydroxy-3,7-dimethyloctanal, 2-methyl-3-(4-tert.-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine and 2-isobutyl-3-methoxypyrazine;

the phenols, phenyl ethers or phenyl esters, such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol and p-cresyl phenylacetat;

the heterocyclic compounds, such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one and 2-ethyl-3-hydroxy-4H-pyran-4-one;

the lactones, such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecandioate; ethylene 1,13-tridecandioate; coumarin; 2,3-dihydrocoumarin and octahydrocoumarin.

Perfume oils according to the invention which comprise one or more compounds of the formula (I) to be used according to the invention as fragrance precursors can be employed for perfumings in the liquid form, undiluted or diluted with a solvent. Suitable solvents for this are e.g. ethanol, isopropanol, diethylene glycol monoethyl ether, glycerol, propylene glycol, 1,2-butylene glycol, dipropylene glycol, diethyl phthalate, triethyl citrate, isopropyl myristate etc.

Perfume oils according to the invention which comprise one or more compounds of the formula (I) according to the invention to be used as fragrance precursors can furthermore be adsorbed on a carrier substance, which ensures both a fine distribution of the fragrances in the product and a controlled release during use. Such carriers can be porous inorganic materials, such as light sulfate, silica gels, zeolites, gypsums, clays, clay granules, gas concrete etc., or organic materials, such as woods and cellulose-based substances.

Perfume oils according to the invention which comprise one or more compounds of the formula (I) to be used according to the invention as fragrance precursors can also be in a microencapsulated or spray-dried form or in the form of an inclusion complex or extrusion product and can be added in this form to the product to be perfumed.

The properties of perfume oils modified in this manner can optionally be optimized further in respect of a more controlled release of fragrance by so-called “coating” with suitable materials, for which purpose waxy plastics, e.g. polyvinyl alcohol, are preferably used.

The microencapsulation of the perfume oils can be carried out, for example, by the co-called coacervation process with the aid of capsule materials e.g. of polyurethane-like substances or soft gelatine. The spray-dried perfume oils can be prepared, for example, by spray drying of an emulsion or dispersion containing the perfume oil, it being possible to use modified starches, proteins, dextrin and plant gums as carrier substances. Inclusion complexes can be prepared e.g. by introducing dispersions of the perfume oil and cyclodextrins or urea derivatives into a suitable solvent, e.g. water. Extrusion products can be achieved by melting the perfume oils with a suitable waxy substance and by extrusion with subsequent solidification, optionally in a suitable solvent, e.g. isopropanol.

In perfume compositions, the amount employed of the compounds of the formula (I) according to the invention to be used as fragrance precursors is 0.01 to 75 wt. %, preferably 0.05 to 50 wt. %, and an amount employed of from 0.5 to 20%, based on the total perfume oil, is particularly preferred.

Perfume oils which comprise the compounds of the formula (I) according to the invention to be used as fragrance precursors can be used in concentrated form, in solutions or in a modified form described above for the preparation of cosmetic care products; in this context in particular for hair care or washing products in which the stability of aldehydes or ketones is low and in which an activation and thus a virtually spontaneous release of the aldehyde or ketone is effected (a) by direct raising of the pH from an acid pH value into the alkaline range with a resulting pH of ≧8.5 or (b) by addition of water. Examples which may be mentioned here are: body care compositions, such as e.g. solid and liquid soaps, bleaching creams, acne creams, hair care products, such as e.g. hair setting lotions, permanent hair-colouring compositions and deodorants and antiperspirants, such as e.g. deodorant and antiperspirant sticks.

Perfume oils which comprise the compounds of the formula (I) according to the invention to be used as fragrance precursors can preferably be employed in concentrated form, in solutions or in a modified form described above for the preparation of hair care products and body care compositions, and in this context in particular for the preparation of permanent hair-colouring compositions.

Perfume oils which comprise the compounds of the formula (I) according to the invention to be used as fragrance precursors can furthermore be employed in concentrated form, in solutions or in a modified form described above for the preparation of e.g. household products, such as floor cleaners, window glass cleaners. bath and sanitary cleaners, solid and liquid WC cleaners. liquid detergents, pulverulent detergents, laundry pretreatment compositions, such as bleaching compositions, soaking compositions and stain removers. detergent tablets, disinfectants and surface disinfectants.

Perfume oils which comprise the compounds of the formula (I) according to the invention to be used as fragrance precursors can preferably be employed in concentrated form, in solutions or in a modified form described above for the preparation of liquid detergents.

The compounds of the formula (I) to be used according to the invention as fragrance precursors can be prepared by methods well-known to the person skilled in the art. The enol esters of the formula (I) wherein R² denotes an (a) branched or unbranched C₁ to C₃ alkyl group or (b) branched or unbranched C₂ to C₃ alkylene group are prepared in accordance with the instructions from D. P. Simmons et al., Helv. Chim. Acta 71, 1000 (1988). The enol esters of the formula (I) wherein R² denotes an (a) branched or unbranched C₄ alkyl group or (b) branched or unbranched C₄ alkylene group are prepared in accordance with the instructions from P. Duhamel et al., J. Chem. Soc. Perkin Trans. 1, 1993, 2509.

The following non-limiting examples illustrate the invention.

### EXAMPLE 1

### Preparation of (E/Z)-acetic acid (2,4-dimethylcyclohex-3-enylidene)methyl ester

2,4-Dimethylcyclohex-3-ene-1-carbaldehyde (13.82 g, 100.0 mmol), sodium acetate (1.54 g, 18.5 mmol) and triethylamine (21.27 g, 210.0 mmol) are initially introduced into acetic anhydride (150 ml) and the mixture is heated at 120° C. for 6 hours. When the reaction has ended (TLC control), the mixture is allowed to cool, the reaction solution is poured into ice-water (100 ml) and the aqueous phase is extracted with ether (150 ml) and cyclohexane (150 ml). The combined organic phases are washed 1× each with 2M NaOH (100 ml) and water (100 ml) and then dried over Na₂SO₄, filtered and evaporated on a rotary evaporator. After fractional distillation (60.0-61.6° C., 0.25 mbar), 17.5 g (E/Z)-acetic acid (2,4-dimethylcyclohex-3-enylidene)methyl ester are obtained as a colourless oil.

E/Z isomer ratio=1:1.

The spectroscopic data correspond to the E isomer:

¹H-NMR (400 MHz, CDCl₃): δ (ppm)=1.10 (d, 7.1 Hz, 3H), 1.64-1.67 (m , 3H), 1.97-2.05 (m, 2H), 2.14 (s, 3H), 2.43 (ddd, J =0.6, 7.2, 13.2 Hz, 1H), 2.49 (ddd, J=0.9, 5.7, 13.2 Hz, 1H), 3.18-3.28 (m, 1H), 5.26-5.31 (m, 1H), 7.00 (q, 1.1 Hz,1 H).

¹³C-NMR (100 MHz, CDCl₃): δ (ppm)=20.7, 20.8, 21.4, 23.3, 30.9, 33.4, 125.5, 126.7, 128.0, 133.3, 168.4.

### EXAMPLE 2

### Preparation of (E/Z)-isobutyric acid (2,4-dimethylcyclohex-3-enylidene)methyl ester

The preparation of (E/Z)-2-methylpropionic acid (2,4-dimethylcyclohex-3-enylidene)methyl ester is carried out analogously to Example 1, isobutyric anhydride having been employed instead of acetic anhydride.

E/Z isomer ratio=1:1.

The spectroscopic data correspond to the E isomer:

¹H-NMR (400 MHz, CDCl₃): δ (ppm)=1.07 (d, 7.1 Hz, 3H), 1.22 (d, J=7.0 Hz, 6H), 1.64-1.67 (m, 3H), 1.98-2.05 (m, 2H), 2.36 (ddd, J=0.7, 7.3, 13.4 Hz, 1H), 2.48 (ddd, J=0.9, 6.1, 13.4 Hz, 1H), 2.62 (sep, J=7.0 Hz, 1H), 3.20-3.26 (m, 1H), 5.26-5.31 (m, 1H), 7.00 (q, 1.1 Hz, 1H).

¹³C-NMR (100 MHz, CDCl₃): δ (ppm)=18.8 (2C), 20.7, 23.5, 23.8, 29.9, 31.3, 34.0, 125.5, 126.6, 127.5, 133.3, 174.1.

### EXAMPLE 3

### Preparation of (E/Z)-pivalic acid (2,4-dimethylcyclohex-3-enylidene)methyl ester

(E/Z)-Acetic acid (2,4-dimethylcyclohex-3-enylidene)methyl ester (26.27 g, 145 mmol) is initially introduced into THF (200 ml), the mixture is cooled to −70° C. and potassium tert-butanolate (24.75 g, 220 mmol), dissolved in THF (100 ml), is added. The mixture is now subsequently stirred at −70° C. for 60 minutes, before pivalic acid chloride (26.57 g, 220 mmol), dissolved in THF (60 ml) is added, and the mixture is then subsequently stirred for another further 120 minutes. When the reaction has ended (TLC control), the reaction solution is poured on to sat. NaHCO₃ solution (250 ml), the phases are separated and the aqueous phase is extracted a further 2× with ether (250 ml). The combined organic phases are dried over Na₂SO₄, filtered and evaporated on a rotary evaporator. The crude product obtained is purified by means of flash chromatography (cyclohexane/EtOAc=60:1, R_{f}=0.23), and 24.50 g of a colourless oil are obtained.

E/Z isomer ratio=2:1.

The spectroscopic data correspond to the E isomer:

¹H-NMR (400 MHz, CDCl₃): δ (ppm)=1.08 (d, 7.1 Hz, 3H), 1.25 (s, 9H), 1.66-1.68 (m, 3H), 1.97-2.05 (m, 2H), 2.38 (ddd, J=0.6, 7.2, 13.2 Hz, 1H), 2.49 (ddd, J=0.9, 5.7, 13.2 Hz, 1H), 3.19-3.28 (m, 1H), 5.27-5.31 (m, 1H), 6.90 (q, 2.2 Hz, 1 H).

¹³C-NMR (100 MHz, CDCl₃): δ (ppm)=21.3, 23.5, 23.8, 27.1 (3C), 30.0, 31.2, 38.8, 125.5, 126.5, 127.7, 133.4, 175.5.

### EXAMPLE 4

### Preparation of (1E/Z)-acetic acid dec-1-enyl ester

The synthesis is carried out analogously to Example 1, decanal having been employed instead of 2,4-dimethylcyclohex-3-ene-1-carbaldehyde. See also P. Z. Bedoukian, J. Am. Chem. Soc. 79, 889-892, (1957).

### EXAMPLE 5

### Preparation of (1E/Z)-isobutyric acid dec-1-enyl ester

The synthesis is carried out analogously to Example 1, decanal having been employed instead of 2,4-dimethylcyclohex-3-ene-1-carbaldehyde and isobutyric anhydride having been employed instead of acetic anhydride.

### EXAMPLE 6

### Preparation of (1E/Z)-pivalic acid dec-1-enyl ester

The synthesis is carried out analogously to Example 3, (1 E/Z)-acetic acid dec-1-enyl ester having been employed instead of (E/Z)-acetic acid (2,4-dimethylcyclohex-3-enylidene)methyl ester.

### EXAMPLE 7

### Preparation of (1E/Z)-acetic acid 3-methyl-5-phenylpent-1-enyl ester

The synthesis is carried out analogously to Example 1,3-methyl-5-phenylpentanal having been employed instead of 2,4-dimethylcyclohex-3-ene-1-carbaldehyde.

### EXAMPLE 8

### Preparation of (1E/Z)-isobutyric acid 3-methyl-5-phenylpent-1-enyl ester

The synthesis is carried out analogously to Example 1,3-methyl-5-phenylpentanal having been employed instead of 2,4-dimethylcyclohex-3-ene-1-carbaldehyde and isobutyric anhydride having been employed instead of acetic anhydride.

### EXAMPLE 9

### Preparation of (1E/Z)-pivalic acid 3-methyl-5-phenylpent-1-enyl ester

The synthesis is carried out analogously to Example 3, (1E/Z)-acetic acid 3-methyl-5-phenylpent-1-enyl ester having been employed instead of (E/Z)-acetic acid (2,4-dimethylcyclohex-3-enylidene)methyl ester.

### EXAMPLE 10

### Preparation of (1E/Z)-acetic acid 3-(4-tert-butyl)-2-methylprop-1-enyl ester

The synthesis is carried out analogously to Example 1,3-(4-tert-butylphenyl)-2-methylpropanal having been employed instead of 2,4-dimethylcyclohex-3-ene-1-carbaldehyde. See also JP 5514137 A1.

### EXAMPLE 11

### Preparation of (1E/Z)-isobutyric acid 3-(4-tert-butyl)-2-methylprop-1-enyl ester

The synthesis is carried out analogously to Example 1,3-(4-tert-butylphenyl)-2-methylpropanal having been employed instead of 2,4-dimethylcyclohex-3-ene-1-carbaldehyde and isobutyric anhydride having been employed instead of acetic anhydride.

### EXAMPLE 12

### Preparation of (1E/Z)-pivalic acid 3-(4-tert-butyl)-2-methylprop-1-enyl ester

The synthesis is carried out analogously to Example 3, (1E/Z)-acetic acid 3-(4-tert-butylphenyl)-2-methylprop-1-enyl ester having been employed instead of (E/Z)-acetic acid (2,4-dimethylcyclohex-3-enylidene)methyl ester.

### EXAMPLE 13

### Preparation of (1E/Z)-acetic acid 3-(1,3-benzodioxol-5-yl)-2-methylprop-1-enyl ester

The synthesis is carried out analogously to Example 1,3-(1,3-benzodioxol-5-yl)-2-methylpropanal having been employed instead of 2,4-dimethylcyclohex-3-ene-1-carbaldehyde.

### EXAMPLE 14

### Preparation of (1E/Z)-isobutyric acid 3-(1,3-benzodioxol-5-yl)-2-methylprop-1-enyl ester

The synthesis is carried out analogously to Example 1,3-(1,3-benzodioxol-5-yl)-2-methylpropanal having been employed instead of 2,4-dimethylcyclohex-3-ene-1-carbaldehyde and isobutyric anhydride having been employed instead of acetic anhydride.

### EXAMPLE 15

### Preparation of (1E/Z)-pivalic acid 3-(1,3-benzodioxol-5-yl)-2-methylprop-1-enyl ester

The synthesis is carried out analogously to Example 3, (1E/Z)-acetic acid 3-(1,3-benzodioxol-5-yl)-2-methylprop-1-enyl ester having been employed instead of (E/Z)-acetic acid (2,4-dimethylcyclohex-3-enylidene)methyl ester.

### EXAMPLE 16

### Preparation of (1E/Z)-acetic acid dodec-1-enyl ester

The synthesis is carried out analogously to Example 1, dodecanal having been employed instead of 2,4-dimethylcyclohex-3-ene-1-carbaldehyde. See also P. Z. Bedoukian, J. Am. Chem. Soc. 79, 889-892, (1957).

### EXAMPLE 17

### Preparation of (1E/Z)-isobutyric acid dodec-1-enyl ester

The synthesis is carried out analogously to Example 1, dodecanal having been employed instead of 2,4-dimethylcyclohex-3-ene-1-carbaldehyde and isobutyric anhydride having been employed instead of acetic anhydride.

### EXAMPLE 18

### Preparation of (1E/Z)-pivalic acid dodec-1-enyl ester

The synthesis is carried out analogously to Example 3, (1E/Z)-acetic acid dodec-1-enyl ester having been employed instead of (E/Z)-acetic acid (2,4-dimethylcyclohex-3-enylidene)methyl ester.

### EXAMPLE 19

### Preparation of (1E/Z)-acetic acid 2,6-dimethylhepta-1,5-dienyl ester

The synthesis is carried out analogously to Example 1,2,6-dimethylhept-5-enal having been employed instead of 2,4-dimethylcyclohex-3-ene-1-carbaldehyde. See also JP 55015433 B4.

### EXAMPLE 20

### Preparation of (1E/Z)-isobutyric acid 2,6-dimethylhepta-1,5-dienyl ester

The synthesis is carried out analogously to Example 1,2,6-dimethylhept-5-enal having been employed instead of 2,4-dimethylcyclohex-3-ene-1-carbaldehyde and isobutyric anhydride having been employed instead of acetic anhydride.

### EXAMPLE 21

### Preparation of (1E/Z)-pivalic acid 2,6-dimethylhepta-1,5-dienyl ester

The synthesis is carried out analogously to Example 3, (1E/Z)-acetic acid 2,6-dimethylhepta-1,5-dienyl ester having been employed instead of (E/Z)-acetic acid (2,4-dimethylcyclohex-3-enylidene)methyl ester.

### EXAMPLE 22

### Preparation of (1E/Z,3E/Z)-acetic acid hexa-1,3-dienyl ester

The synthesis is carried out analogously to Example 1, (3E)-hex-3-enal having been employed instead of 2,4-dimethylcyclohex-3-ene-1-carbaldehyde. See also B. M. Trost et al. J. Am. Chem. Soc. 100, 3930-3931, (1978).

### EXAMPLE 23

### Preparation of (1E/Z,3E/Z)-isobutyric acid hexa-1,3-dienyl ester

The synthesis is carried out analogously to Example 1, (3E)-hex-3-enal having been employed instead of 2,4-dimethylcyclohex-3-ene-1-carbaldehyde and isobutyric anhydride having been employed instead of acetic anhydride.

### EXAMPLE 24

### Preparation of (1E/Z,3E/Z)-pivalic acid hexa-1,3-dienyl ester

The synthesis is carried out analogously to Example 3, (1E/Z,3E/Z)-acetic acid hexa-1,3-dienyl ester having been employed instead of (E/Z)-acetic acid (2,4-dimethylcyclohex-3-enylidene)methyl ester.

### EXAMPLE 25

### Preparation of (1E/Z,5Z)-acetic acid octa-1,5-dienyl ester

The synthesis is carried out analogously to Example 1, (5Z)-oct-5-enal having been employed instead of 2,4-dimethylcyclohex-3-ene-1-carbaldehyde.

### EXAMPLE 26

### Preparation of (1E/Z,6Z)-acetic acid nona-1,6-dienyl ester

The synthesis is carried out analogously to Example 1, (6Z)-non-6-enal having been employed instead of 2,4-dimethylcyclohex-3-ene-1-carbaldehyde.

### EXAMPLE 27

### Preparation of (1E/Z)-acetic acid 3-(4-isopropylphenyl)-2-methylprop-1-enyl ester

The synthesis is carried out analogously to Example 1,3-(4-isopropylphenyl)-2-methylpropanal having been employed instead of 2,4-dimethylcyclohex-3-ene-1-carbaldehyde. See also U.S. Pat. No. 3,023,247.

### EXAMPLE 28

### Preparation of (1E/Z)-acetic acid 2,610-trimethylundeca-1,9-dienyl ester

The synthesis is carried out analogously to Example 1,2,6,10-trimethylundec-9-enal having been employed instead of 2,4-dimethylcyclohex-3-ene-1-carbaldehyde.

The compounds of the formula (I) to be used according to the invention as fragrance precursors were incorporated into numerous consumer products and the use properties thereof were investigated by various methods. In the preparation of the formulations for the consumer products, molar equivalents of the aldehydes or ketones were employed on the one hand in the form of the enol esters and on the other hand in the form of the free aldehydes or ketones, in order to ensure a comparability.

Method 1: Storage Stability

The storage stability of a fragrance or a fragrance precursor is defined as the percentage amount of the substance still present after storage.

$$\frac{Amount after storage}{Amount before storage} * 100 \% = Storage stability [ \% ]$$

For determination and for comparison of the storage stability, both the fragrance precursor and the corresponding aldehydes or ketones are incorporated into separate samples of the same formulation of a consumer product, such as e.g. hair-colouring composition or soap. The separate samples are then divided into portions. The one portion of the samples is subjected immediately to a suitable extraction and an analytical measurement, in order to determine the amount of fragrance precursor or aldehyde or ketone before the storage. In the analysis by e.g. gas chromatography, a suitable standard is used for quantification. The second portion is subjected to storage at elevated temperature for a defined time and then extracted and quantified using the same methods.

### EXAMPLE 29

### Permanent Hair-Colouring Composition

(a) Stability in the Developer Composition:

The formulation of the developer composition typically comprises water, hydrogen peroxide, acids, such as e.g. phosphoric acid, citric acid etc., thickeners, emulsifiers, preservatives, complexing agents, silicones, solvents and further auxiliary substances.

The fragrance precursors (see Table 1) are added to batches of the developer formulation in a dosage of 1% and the mixtures are stored at 40° C. for one month.

TABLE 1
Storage stability of fragrance precursors in the developer
composition

| | Stability | Stability [%] | Stability [%] |
|---|---|---|---|
| Fragrance precursor | [%]0 days | 13 days | 28 days |
| (1E/Z)-Acetic acid dec-1-enyl | 100 | 100 | 98 |
| ester | | | |
| (1E/Z)-Acetic acid 3-methyl-5- | 100 | 100 | 100 |
| phenylpent-1-enyl ester | | | |
| (1E/Z)-Acetic acid 3-(4-tert- | 100 | 100 | 97 |
| butyl)-2-methylprop-1-enyl ester | | | |
| (E/Z)-Isobutyric acid (2,4- | 100 | 100 | 100 |
| dimethylcyclohex-3- | | | |
| enylidene)methyl ester | | | |
| (1E/Z)-Isobutyric acid 3-(1,3- | 100 | 100 | 100 |
| benzodioxol-5-yl)-2-methylprop- | | | |
| 1-enyl ester | | | |
| (1E/Z)-Isobutyric acid dodec-1- | 100 | 100 | 100 |
| enyl ester | | | |
| (E/Z)-Pivalic acid (2,4- | 100 | 100 | 100 |
| dimethylcyclohex-3- | | | |
| enylidene)methyl ester | | | |
| (1E/Z)-Pivalic acid 3-(4-tert- | 100 | 100 | 100 |
| butyl)-2-methylprop-1-enyl ester | | | |
| (1E/Z)-Pivalic acid dodec-1-enyl | 100 | 100 | 100 |
| ester | | | |

The fragrance precursors are stable in terms of colour, smell and analysis over the period of one month.

(b) Rate of Release During Colouring of Hair:

For determination of the rate of hydrolysis, various fragrance precursors (see Table 2) were added to the developer composition in a concentration of in each case 0.3%. The ammoniacal colouring composition, which is composed of 2 to 16% ammonia and/or substitutes, such as e.g. alkanolamines, in particular monoethanolamine, water, thickeners, emulsifier, consistency-imparting agents, reactive dyestuffs, solvents, complexing agents, stabilizers and preservatives, is then added to the developer composition in the ratio of 1:1. Samples are now taken from the hair-colouring composition at defined intervals of time, the samples are neutralized immediately and extracted with solvent and the content of fragrance precursor and of aldehyde or ketone released is determined by means of gas chromatography using an internal and external standard.

TABLE 2
Analytically determined release of aldehyde during colouring of
hair

| | Maximum aldehyde concentration | | | | |
|---|---|---|---|---|---|
| | in [%] after | | | | |
| Aldehydes | 1 min | 5 min | 10 min | 20 min | 30 min |
| 2,4-Di-methylcyclohex-3-ene-1-carbaldehyde | 65 | 100 | 100 | 75 | 70 |
| released from Example 1 | | | | | |
| 3-(4-tert-Butylphenyl)-2-methylpropanal | 29 | 91 | 100 | 83 | 80 |
| released from Example 10 | | | | | |
| 3-Methyl-5-phenylpentanal released from | 90 | 100 | 87 | 87 | 84 |
| Example 7 | | | | | |
| Decanal released from Example 4 | 13 | 92 | 100 | 92 | 84 |
| 3-(1,3-Benzodioxol-5-yl)-2-methylpropanal | 29 | 100 | 91 | 85 | 82 |
| released from Example 13 | | | | | |

The fragrance precursors in the formulation for colouring hair showed a virtually spontaneous hydrolysis to the corresponding aldehydes after combination of the developer composition with the ammoniacal hair-colouring solution. Virtually 100% of the maximum aldehyde concentration had already formed from all the fragrance precursors after 5 minutes.

(c) Evaluation of Smell:

The smell intensity of the individual aldehydes released was determined sensorially from the hair strands. The scale of the sensorial intensity extends from 1.0=no smell up to 9.0=very intense. The developers were prepared analogously to the experiment described under (b), and after the individual developers had been brought together with the ammoniacal colouring composition, the resulting hair-colouring composition was applied to the hair strands and the intensity of the smell of the aldehydes released was determined by a panel (5 persons) after certain intervals of time.

TABLE 3
Sensorially determined aldehyde intensity during colouring
of hair

| | Aldehyde intensity after | | | | | | |
|---|---|---|---|---|---|---|---|
| Aldehydes | 2 min | 4 min | 6 min | 8 min | 10 min | 15 min | 30 min |
| 2,6-Dimethylhept-5-enal released from | 5.5 | 5.7 | 5.7 | 5.5 | 5.3 | 5.3 | 5.0 |
| Example 19 | | | | | | | |
| Dodecanal released from Example 16 | 5.2 | 5.0 | 4.8 | 4.5 | 3.9 | 3.3 | 3.0 |
| Decanal released from Example 4 | 5.7 | 6.0 | 6.2 | 6.0 | 5.0 | 4.6 | 4.3 |
| (6Z)-Non-6-enal released from Example 26 | 5.2 | 4.7 | 4.0 | 3.8 | 3.7 | 3.9 | 3.3 |
| (SZ)-Oct-5-enal released from Example 25 | 7.0 | 7.2 | 6.8 | 6.8 | 6.7 | 5.9 | 4.8 |

The fragrance precursors in the formulation for colouring hair showed a virtually spontaneous hydrolysis to the corresponding aldehydes after combination of the developer composition with the ammoniacal hair-colouring solution. The aldehydes released had already reached their maximum intensity after 4 minutes.

This shows, surprisingly, a considerable advantage in the use of the compounds of the formula (I) to be used according to the invention as fragrance precursors for the controlled release of aldehydes or ketones in perfume oils for alkaline hair-colouring compositions.

### EXAMPLE 30

### Soap

The following soap formulation can be prepared by generally known methods. The data relate to percentages by weight. The soaps A and B obtained were used for washing and analysed both directly and after storage for four weeks.

TABLE 4
Soap formulation

| Constituents | | A | B |
|---|---|---|---|
| Soap base | Sodium Tallowate | 60.0 | 60.0 |
| Soap base | Sodium Cocoate | 27.0 | 27.0 |
| | Glycerine | 2.0 | 2.0 |
| | Sodium Chloride | 0.5 | 0.5 |
| Stabilizer | Tetrapotassium Etidronate | 0.3 | 0.3 |
| Stabilizer | Tocopherol | 0.1 | 0.1 |
| Colouring agent | Titanium Dioxide | 0.1 | 0.1 |
| | Water | 7.0 | 7.0 |
| | Diethyl Phthalate (DEP) | 2.4 | 2.4 |
| Example 5 | (1E/Z)-Isobutyric acid dec-1- | 0.60 | |
| | enylester | | |
| | n-Decanal | | 0.60 |

(a) Evaluation of Smell and Colour:

Soap formulations A and B were stored at room temperature for approx. three months.

Soap A according to the invention, which comprises the fragrance precursor (1E/Z)-isobutyric acid dec-1-enyl ester, showed no change or only a slight change in colour, while (comparison) soap B had a yellowish or grey discoloration. A high colour stability is thus achieved by the use of the fragrance precursor.

After storage, in each case 1 g of the soaps was dissolved in 100 g of hand-hot water, and the pieces of soap were used for washing the skin.

In all cases the fragrance impression above the aqueous solution of soap A according to the invention, which comprises the fragrance precursor, was significantly more intense than the fragrance impression of soap B, which comprises the free aldehyde.

The fragrance impression of the washed skin which was washed with soap A was likewise higher than the fragrance impression after washing with soap B.

(b) Storage Stability:

Soap formulations A and B were stored at room temperature in the dark for approx. one month. The depot preparation in soap A according to the invention showed a significantly higher storage stability than the corresponding aldehyde in (comparison) soap B.

1. A method for enhancing a composition with a fragrance precursor which can be activated to release one or more organoleptically active compounds, said method comprising adding to said composition a compound of the formula I 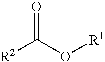
in which
R¹ is the radical (a) of the enol form of an aldehyde having 6 or more C atoms or (b) of a ketone having 10 or more C atoms, and
R² is an (a) branched or unbranched C₁ to C₄ alkyl group or (b) branched or unbranched C₂ to C₄ alkylene group.

2. The method according to claim 1, wherein R² is chosen from the group consisting of methyl, ethyl, n-propyl, iso-propyl, n-butyl, sec-butyl, iso-butyl and tert-butyl, ethenyl, methylethenyl, 1-propenyl, 2-propenyl, 2-methyl-1-propenyl, 1-methyl-1-propenyl, 1-butenyl and 3-butenyl.

3. The method according to claim 2, wherein R² is chosen from the group consisting of methyl, ethyl, n-propyl and iso-butyl, ethenyl, methylethenyl, 1-propenyl, 2-methyl-1-propenyl and 1-methyl-1-propenyl.

4. Method for the release of a fragrance, having the following steps:
adding to a composition a fragrance precursor formulation comprising a fragrance precursor compound according to the following formula and a medium in which said compound is stable 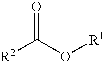
in which
R¹ is the radical (a) of the enol form of an aldehyde having 6 or more C atoms or (b) of a ketone having 10 or more C atoms, and
R² is an (a) branched or unbranched C₁ to C₄ alkyl group or (b) branched or unbranched C₂ to C₄ alkylene group,
treating said formulation such that the fragrance precursor compound dissociates and releases one or more organoleptically active compounds.

5. Method according to claim 4, wherein the medium (a) is acidic and oxidative, or (b) is alkaline and has a water content of ≦10 wt. %, based on the total weight of the medium.

6. Method according to claim 5, wherein the treatment of the formulation comprises
when said medium is acidic and oxidative, raising the pH of the formulation to a value of ≧8.5, or
when said medium is alkaline, raising the water content of the formulation to >10 wt. %.

7. Method according to claim 5, wherein the formulation
when said medium is acidic and oxidative, is chosen from the group which consists of: a developer composition for a permanent hair-colouring composition, permanent wave fixing composition, bleaching cream, acne cream, sanitary cleaner and surface cleaner, or
when said medium is alkaline, is chosen from the group which consists of liquid detergents for packages in water-soluble film, deodorant or antiperspirant sticks and soaps.

8. Cosmetic, washing and/or cleaning formulation comprising:
less than 1 wt. %, based on total formulation weight, of a fragrance precursor compound of the following formula: 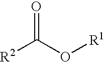
in which
R¹ is the radical (a) of the enol form of an aldehyde having 6 or more C atoms or (b) of a ketone having 10 or more C atoms, and
R² is an (a) branched or unbranched C₁ to C₄ alkyl group or (b) branched or unbranched C₂ to C₄ alkylene group, and
a medium in which said compound is stable.

9. Formulation according to claim 8, wherein the medium (a) is acidic and oxidative, or (b) is alkaline and has a water content of ≦10 wt. %, based on the total weight of the medium.

10. Formulation according to claim 9, wherein:
the formulation is acidic and oxidative and is chosen from the group which consists of: developer composition for a permanent hair-colouring composition, permanent wave fixing composition, bleaching cream, acne cream, sanitary cleaner and surface cleaner, or
the formulation is alkaline and has a water content of ≦10 wt. % and is chosen from the group which consists of: liquid detergents for packages in water-soluble film, deodorant or antiperspirant sticks and soaps.

11. Formulation according to claim 8, wherein
(a) the fragrance precursor compound is dispersed or dissolved in the medium, and/or
(b) the fragrance precursor compound is employed as a constituent of a perfume oil which is dispersed or dissolved in the medium.

12. Perfume oil comprising
at least 0.1 wt. % based on the total weight of the perfume oil of a fragrance precursor compound of the formula I 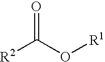
in which
R¹ is the radical (a) of the enol form of an aldehyde having 6 or more C atoms or (b) of a ketone having 10 or more C atoms, and
R² is an (a) branched or unbranched C₁ to C₄ alkyl group or (b) branched or unbranched C₂ to C₄ alkylene group, and
one or more fragrances.

13. A formulation according to claim 11 wherein said formulation is (i) an adsorbed on a carrier, (ii) microencapsulated, (iii) spray-dried, (iv) in an inclusion complex, (v) in an extruded carrier, or (vi) coated on a carrier.

14. A perfume oil according to claim 12 wherein said perfume oil is (i) an adsorbed on a carrier, (ii) microencapsulated, (iii) spray-dried, (iv) in an inclusion complex, (v) in an extruded carrier, or (vi) coated on a carrier.
